(19) United States
(12) Patent Application Publication
(10) Pub. No.: US 2021/0059935 A1
(43) Pub. Date: Mar. 4, 2021

(54) SELF-EMULSIFYING NANO-EMULSIONS

(71) Applicant: MEDPHARM HOLDINGS, LLC, Denver, CO (US)

(72) Inventor: Scott Karolchyk, Denver, CO (US)

(21) Appl. No.: 17/011,492

(22) Filed: Sep. 3, 2020

Related U.S. Application Data
(60) Provisional application No. 62/895,780, filed on Sep. 4, 2019.

(51) Int. Cl. A61K 9/107; A61K 47/26; A61K 47/24; A61K 47/44; A61K 47/14; A61K 36/185

(52) U.S. Cl. CPC A61K 9/1075; A61K 47/26; A61K 47/24; A61K 9/0053; A61K 47/14; A61K 36/185; A61K 47/44

(57) ABSTRACT

The invention relates to the preparation of a hydrophilic edible emulsified drug delivery system (SENE) which presents poorly soluble medicaments in a solubilized dispersion. The SENE further includes a combination of surfactants having an HLB of between 8-15 and a solvent/stabilizer. The SENE has practically no taste and yields highly translucent and fully water-compatible nano-emulsions.

### CROSS-REFERENCE TO RELATED APPLICATIONS

This application claims the benefit of priority to U.S. Provisional Patent Application No. 62/895,780, filed Sep. 4, 2019, entitled “SELF-EMULSIFYING NANO-EMULSIONS,” the entire disclosure of which is hereby incorporated by reference in its entirety.

### FIELD OF THE INVENTION

This invention relates to the preparation of water-soluble, self-emulsifying cannabinoids in a tasteless and translucent nano-emulsion (SENE).

### BACKGROUND OF THE INVENTION

Cannabinoids are diverse chemical compounds that naturally occur in the cannabis plant and act on cannabinoid receptors CB1 and CB2. Cannabis plants contain hundreds of different cannabinoids, including the major cannabinoids cannabidiol (CBD), tetrahydrocannabinol (THC), cannabinol (CBN), and cannabigerol (CBG).

Cannabis has been used for medicinal purposes for thousands of years. Its active compounds produce pharmacological effects throughout the body, especially in the central nervous system and the immune system. There presently exists the need to provide more effective and safer cannabis dosage forms for various medical uses, including treatment of pain, nausea, spasticity in multiple sclerosis, side effects of chemotherapy, and various other medical conditions.

Even though THC and CBD possess a wide range of activities, the administration of these components has been hampered by their extreme water insolubility and poor bioavailability, largely due to the highly lipophilic nature of cannabinoids. Formulation of cannabinoids as aerosols, sprays, eye drops, etc. requires aqueous solutions of these highly lipophilic active compounds.

Various strategies have been used to overcome problems associated with oral and topical absorption and bioavailability of poorly soluble drugs, such as particle size reduction, complexation with cyclodextrins (CDs), salt formation, solid dispersions, use of surfactants, nanoparticles, nanocarriers, nano-emulsions, prodrug formation, etc. Translucent nano-emulsions are hard to design as they require optimized carrier oil and surfactant formulations and well-adjusted ultrasonic exposure parameters (amplitude, temperature, exposure time, etc.) However, none of these strategies have proven satisfactory for various reasons. For example, CDs are very expensive and difficult to work with. For these and other reasons, there is a need for the present invention.

### SUMMARY OF THE INVENTION

The invention provides water soluble cannabinoids in a self-emulsifying, tasteless, and translucent stabilized nano-emulsion (SENE) which has use as an internal or external carrier for oral and other forms of delivery of poorly water-soluble medicaments including cannabis, terpenes and minor cannabinoids. In one embodiment of the invention, the SENEs comprise a mixture of oleate surfactants having an HLB between 8-15, such as polyoxyethylene-sorbitan-20 mono-oleate (Polysorbate 80 or Tween 80) and sorbitan monooleate (Span 80), a solvent such as ethoxy diglycol, a vegetable or edible oil such as caprylic acid triglyceride, and water. In another embodiment of the invention, the SENEs comprise a mixture of surfactants having an HLB between 8-15, such as non-ionic and anionic lecithins and Polysorbate 80, a stabilizer such as polyethoxythylene-660-12-hydroxystearate (Kolliphor HS15), a vegetable or edible oil such as caprylic acid triglyceride, and water.

These combinations stabilize the actives, provides complete water solubility of the cannabinoids, and further stabilizes the final products. In addition, the SENEs enhance the bioavailability of the cannabinoids and accelerate their onset of action to ensure precise and reproducible therapeutic dosing, with an effect in 15 minutes (fasted) or 30 minutes (fed), maximum effect of less than an hour for a duration of 5 hours.

In another embodiment of the invention, the invention provides an all-in-one combination system that tremendously simplifies the ultrasonic production of high-quality, translucent nano-emulsions of bio-active ingredients, such as cannabis extracts. This product is for manufacturers without the capability or desire to develop their own formulations and processing procedures. This product is designed to work in conjunction with a laboratory, bench or industrial ultrasonic processor, and comes with detailed, easy-to-follow instructions. It has practically no taste of its own and yields highly translucent and fully water-compatible nano-emulsions with droplet sizes less than 50 nanometers, ensuring a high bioavailability, accelerated onset of action, and permanent product stability.

Loaded with up to concentrations of 100 mg/ml of cannabinoids and/or other active ingredients, these nano-emulsions can be easily sterilized by filtration and infused into a variety of water-based products without changing their appearance. Finished products include CBD and THC-infused beverages (water, tea, coffee, beer, juice, etc.), creams, oral and nasal sprays, ophthalmics, injections, tinctures, tablets, powders, edibles, thin film oral strips and many more.

Other features and advantages of the invention will be apparent from and are encompassed by the following detailed description and claims.

### DETAILED DESCRIPTION OF THE PREFERRED EMBODIMENT

The present invention relates to the use of a combination of surfactants having an HLB of between about 8-15 and a solvent stabilizer system in a unique ratio to provide high-quality, translucent nano-emulsions of bio-active ingredients, such as cannabis extracts. The product yields highly translucent and fully water-compatible nano-emulsions with droplet sizes less than 50 nanometers (mean 35 nm), ensuring a high bioavailability, accelerated onset of action, and permanent product stability.

One embodiment of the invention involves the use of a Span and Polysorbate surfactant combination having an HLB of between about 8-15 with a solvent such as ethoxy glycol (EG). Such compositions are not preferred for oral use as they typically have a bitter taste. Another embodiment of the invention involves the use of a lecithin and Polysorbate surfactant combination having an HLB of between about 8-15 with a solvent system such as polyoxyethyene-660-12-hydroxystearate (Kolliphor HS15). Such compositions are tasteless and are therefore preferred for oral use.

Traditionally, lecithins or phospholipids are emulsifiers of choice for producing an oil in water (O/W) nanosized emulsion. However, additional emulsifiers preferably dissolved in the aqueous phase are usually included in the emulsion composition, such as mixtures of glycerophospholipids, phosphatidylcholine, phosphatidylethanolamine, phosphatidylserine and phosphatic acid. These additional emulsifiers and stabilizing agents impart a strong, bitter, soapy taste to the finished emulsion, which is unacceptable for cannabis edibles and other edible products and beverages. It has not been previously known to formulate cannabis with a non-ionic emulsifier and a non-ionic stabilizer. Lecithin phospholipids are available from several sources including soya, egg, sunflower and anionic forms such as Lipoid S75.

Polyoxyethlene-660-12-hydroxystearate (Kolliphor HS15) is a graft polymer frequently used as a stabilizer in topical formulations (dermal drug delivery systems), transdermal delivery systems, parenteral, ocular and intranasal delivery systems. It provides a steric stabilization mechanism and is a great solubilizer. It is also used as a novel ingredient in emulsions, sunless tanning products, and in a wide range or hair-coloring products. It has not previously been used in SENE form to emulsify active medications such as cannabinoids. It keeps the acceptable nanoemulsion properties intact, protecting the emulsion from harsh conditions such as high temperature, shear stresses and ionic medication loads. Chemical names for Kolliphor HS15 include Solutol HS15, polyoxyl 15 hydroxystearate, and macrogol 660 hydroxystearate.

Polysorbate 80 (C₆₄H₁₂4O₂₆) is a common excipient and solubilizing agent used in the pharmaceutical industry. Polysorbate 80 (also known as polyoxyethylene-sorbitan-20 mono-oleate, or Tween 80) is used in the pharmaceutical and cosmetic industry in lotions, medical preparations (e.g., vitamin oils, vaccines, and intravenous preparations) and as an excipient in tablets. The full chemical names for polysorbate 80 are polyoxyethylene (20) sorbitan monooleate and (x)-sorbitan mono-9-octadecenoate poly(oxy-1,2-ethanediyl), and is also known under the brand names of Alkest TW 80, Scattics, Canarcel, Poegasorb 80, and Tween 80.

Ethoxy diglycol (EG), also known as diethylene glycol monoethyl ether, Carbitol, transcutoldiethylene glycol monoethyl ether, is a liquid which has a long history of use in cosmetic and over-the-counter topically applied products. EG has a long history of use in formulations as a co-surfactant and a penetration enhancer. It is frequently utilized in topical formulations (dermal drug delivery systems), transdermal delivery systems, ocular and intranasal delivery systems. It is also used as an ingredient in nanoemulsions, sunless tanning products, and in a wide range or hair-coloring products. It has not previously been used in SENE form to emulsify active medications such as cannabinoids.

The nanoemulsions of the invention preferably include EG or other solvent, such as poloxamer 188 and 407. However, many other solvents are appropriate for use in the invention including, but not limited to the following categories:
water-soluble organic solvents, such as polyethylene glycol 300, polyethylene glycol 400, ethanol, propylene glycol, glycerin, N-methyl-2-pyrrolidone, dimethylacetamide, and dimethylsulfoxide);
non-ionic surfactants (Cremophor EL, Cremophor RH 40, Cremophor RH 60, d-α-tocopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, and mono- and di-fatty acid esters of PEG 300, 400, or 1750);
water-insoluble lipids, such as castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil), organic liquids/semi-solids (beeswax, d-α-tocopherol, oleic acid, medium-chain mono- and diglycerides);
various cyclodextrins (α-cyclodextrin, β-cyclodextrin, hydroxypropyl-β-cyclodextrin, and sulfobutylether-β-cyclodextrin); and
phospholipids (hydrogenated soy phosphatidylcholine, distearoylphosphatidylglycerol, 1-α-dimyristoylphosphatidylcholine, 1-α-dimyristoylphosphatidylglycerol).

Polysorbate 80 (C₆₄H₁₂4O₂₆) is a common excipient and solubilizing agent used in the pharmaceutical industry. Polysorbate 80 (also known as polyoxyethylene-sorbitan-20 mono-oleate, or Tween 80) is used in the pharmaceutical and cosmetic industry in lotions, medical preparations (e.g., vitamin oils, vaccines, and intravenous preparations) and as an excipient in tablets. The full chemical names for polysorbate 80 are polyoxyethylene (20) sorbitan monooleate and (x)-sorbitan mono-9-octadecenoate poly(oxy-1,2-ethanediyl), and is also known under the brand names of Alkest TW 80, Scattics, Canarcel, Poegasorb 80, and Tween 80.

Span 80, Sorbitan Monooleate, NF is used as an emulsifier and nonionic surfactant in pharmaceutical formulations. Sorbitan esters are widely used as W/O emulsifiers and, when used in combination with ethoxylated sorbitan esters (the polysorbate range), they contribute to the overall stability of O/W emulsions.

Manipulation of the Span 80/Polysorbate 80 ratio produces emulsifying systems of various HLB values, allowing the emulsification of a wide range of oils and waxes. Span 80 is a liquid W/O emulsifier and O/W emulsion stabilizer particularly recommended for use with unsaturated lipid components such as highly purified coconut or vegetable oils. Common and brand names of Span 80 include sorbitan monooleate, Arlacel 80, Sorbitan, mono-(9Z)-9-octadecenoate, sorbitan oleate, UNII-06XEA2VD56, 06XEA2VD56, NCGC00164240-01, DSSTox_CID_7397, DSSTox_RID_78437, DSSTox_GSID_27397, Glycomul O, Sorbitan O, Alkamuls SMO, Armotan MO, Dehymuls SMO, Lonzest SMO, Kosteran O 1, Crill 4, Sorbester P 17. Disponil 100, Montan 80, Newcol 80, Nonion OP80R, Sorgen 40, Sorgen 40A, Montane 80 VGA, Radiasurf 7155. Rheodol AO 10, Atmer 05, Emasol 410, Emasol O 10, Emasol O 10F, Kemmat S 80, Nikkol SO 10, Nikkol SO-15, Rheodol SP-O 10, Rikemal O 250, Sorbitan, mono-9-octadecenoate, (Z)-, and Sorbon S 80.

The EG or other solvent and oleate surfactants of the invention are placed in a SENE drug delivery system which presents the cannabinoids (or other water insoluble drug) in a solubilized dispersion. The model nanoemulsions of the invention preferably comprise caprylic acid triglyceride as the oil but the invention may generally include vegetable oils having an iodine value between 70 and 110 including, but not limited to, soybean oil, sunflower oil, maize germ oil, olive oil, peanut oil, etc. Besides unsaturated fatty acid triglyceride esters, their admixtures with sucrose esters containing 3-8 fatty acid radicals per sucrose molecule may also be used in the nanoemulsions of the invention.

The hydroxystearate, polysorbate, and lecithin surfactants of the invention are placed in a SENE drug delivery system which presents the cannabinoids (or other water insoluble drug) in a solubilized dispersion. The model nanoemulsions of the invention preferably comprise caprylic acid triglyceride as the oil but the invention may generally include vegetable oils having an iodine value between 70 and 110 including, but not limited to, medium chain triglycerides, long chain triglycerides, squalene, soybean, sesame, corn, olive, sunflower, flax, avocado. Besides unsaturated fatty acid triglyceride esters, their admixtures with sucrose esters containing 3-8 fatty acid radicals per sucrose molecule may also be used in the nanoemulsions of the invention.

The SENEs further comprise a combination of oleate surfactants to provide an HLB of between about 8-15. As already noted, in one embodiment of the invention a convenient pair of oleate surfactants for this purpose is Polysorbate 80 (HLB=15) and Span 80 (HLB=4.3), resulting in a translucent emulsion with an HLB of about 12. Mean droplet sizes of this particular nano-emulsion are between about 30 nm-180 nm depending on the ultrasonic amplitude and processing rate utilized. The amplitudes ranges from 30-150 microns and the processing rate ranges from 4-20 ml/min. Translucent emulsions formed with this combination have a mean droplet size (MDS) of less than 35 nm and a distillate concentration of about 5% or less. Emulsions of the invention having a distillate concentration of greater than about 5% are white in nature, having the appearance of skim milk. It is to be understood that other Polysorbate/Span combinations which may include Polysorbate 20, Span 20, etc., may also be useful in the invention so long as it results in an HLB within the range of 8-15.

In another embodiment of the invention a mixture of surfactants is lecithin (HLB=8) and Polysorbate 80 (HLB=15), and a stabilizer such as Kolliphor HS15, resulting in a translucent emulsion with an HLB of about 9. Mean droplet sizes of this particular nano-emulsion are between about 30 nm-180 nm depending on the ultrasonic amplitude and processing rate utilized. The amplitudes ranges from 30-150 microns and the processing rate ranges from 4-20 ml/min. Translucent emulsions formed with this combination have a mean droplet size (MDS) of less than 35 nm and a distillate concentration of about 5% or less. Emulsions of the invention having a distillate concentration of greater than about 5% are white in nature, having the appearance of skim milk.

As previously noted, the SENEs may be used to solubilize one or more poorly water soluble drugs, including cannabis. The cannabis extracts of the invention are any that can be derived or extracted from cannabis plants. Cannabis plants produce a unique family of terpeno-phenolic compounds called cannabinoids, which produce the “high” one experiences from consuming marijuana. There are 483 identifiable chemical constituents known to exist in the cannabis plant, and at least 85 different cannabinoids have been isolated from the plant. The two cannabinoids usually produced in greatest abundance are cannabidiol (CBD) and/or (−)-trans-Δ⁹-tetrahydrocannabinol (THC), but only THC is psychoactive.

Cannabis plants are categorized by their chemical phenotype or “chemotype,” based on the overall amount of THC produced, and on the ratio of THC to CBD. Although overall cannabinoid production is influenced by environmental factors, the THC/CBD ratio is genetically determined and remains fixed throughout the life of a plant. Non-drug plants produce relatively low levels of THC and high levels of CBD, while drug plants produce high levels of THC and low levels of CBD. Besides CBD and THC, other cannabinoids include, but are not limited to, cannabichromene (CBC), cannabigerol (CBG) cannabinidiol (CBND), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM). Cannabinoids are derived from their respective 2-carboxylic acids (2-COOH) by decarboxylation (catalyzed by heat, light, or alkaline conditions). As a general rule, the carboxylic acids form of the cannabinoid have the function of a biosynthetic precursor.

As noted, the present invention relates to the solubilization of use of any cannabis plant extract in any form. In addition to cannabinoids, cannabis plants produce terpenes, a diverse group of organic hydrocarbons that are the building blocks of the cannabinoids. Over 100 different terpenes have been identified in the cannabis plant, and every strain tends toward a unique terpene type and composition. The terpenes act synergistically with the cannabinoids to provide a therapeutic effect. Examples of some common terpenes found in cannabis include borneol, caryophyllen, cineole/eucalyptol, delta3carene, limonene, linolool, myrcene, pinene, and pulegone.

The cannabinoid extract starting materials are typically mixtures of at least 95% total cannabinoids which include terpenes and/or flavonoids. Preferably the extracts contains a mixture of at least cannabinoids four cannabinoid such as tetrahydrocannabinolic acid (THCa), cannabidiolic acid (CBDa), cannabinolic acid (CBNa) cannabichromenic acid (CBCa), tetrahydrocannabinol (THC), cannabinol (CBN), cannabidiol (CBD) and cannabichromene (CBC). The terpene and/or flavonoids in the extract include, but are not limited to, myrcene, alpha-bisabolol, caryophyllene, limonene, eucalyptol, nerolidol, terpineol, caphene, valencene, geraniol, humulene, delta-3-carene, borneol, alpha-pinen and beta-pinene, and linalool.

Therefore, in a further aspect the invention provides a method of making a SENE nanoemulsion composition preferably comprising, as an active agent, a substance which is an extract from at least one cannabis plant variety. The invention, however, may also be used to solubilize any water-insoluble active including, but not limited to, all components of marijuana and hemp, essential oils, itraconzole and other water insoluble active drugs. Separate extracts may be prepared from single cannabis plant varieties having differing cannabinoid content (e.g. high THC and high CBD plants) and then mixed or blended together prior to formulation to produce the final pharmaceutical composition. This approach is preferred if, for example, it is desired to achieve a defined ratio by weight of individual cannabinoids in the final formulation. Alternatively, plant material from one or more cannabis plant varieties of defined cannabinoid content may be mixed together prior to extraction of a single botanical drug substance having the desired cannabinoid content, which may then be formulated into a final pharmaceutical composition.

Water may also be added to the compositions of the invention in an amount sufficient to bring the composition to the desired volume. The water may be of any type, including tap, distilled, ionized, etc.

A preferred formulation includes a cannabinoid mixture where THC is greater than or equal to 95%; a CBD is less than 1%; CBN is less than 3%; and CBC is less than 1%. In some aspects the formulation further includes d-limonene, linalool, 1,8-cineole (eucalyptol), alpha-pinene, terpineol-4-ol, p-cymene, borneol, delta-3-carene, beta-sitosterol, cannflavin A, apigenin, and quercetin.

Another preferred formulation includes a cannabinoid mixture where the THC is less than or equal to 35%; CBD is greater than or equal to 60%; THC is less than 1%; CBN is less than 1%; and CBC is less than 1%. In some aspects the formulation further includes d-limonene, linalool, 1,8-cineole (eucalyptol), alpha-pinene, terpineol-4-ol, p-cymene, borneol, delta-3-carene, beta-sitosterol, cannflavin A, apigenin, and quercetin.

In yet another preferred embodiment the formulation includes a cannabinoid mixture where the THC is greater than or equal to 40%; CBD is greater than or equal to 40%; THC is less than 1%; CBN is less than 1%; and CBC is less than 1%. In some aspects the formulation further includes beta-myrcene, beta-caryophyllene, pulegone, alpha-terpineol, beta-sitosterol, cannflavin A, apigenin, and quercetin.

In accordance with the methods of the invention, in one embodiment, the cannabinoid extract or other poorly water soluble medicament is combined with the triglyceride oil, Span 80, Polysorbate 80, EG, and water. In another embodiment, the cannabinoid extract or other poorly water soluble medicament is combined with the triglyceride oil, lecithin, Polysorbate 80, Kolliphor HS15 and water. The mixture may optionally be stirred/agitated to more thoroughly combine the ingredients. In one embodiment, the mixture undergoes ultrasonication until the ingredients are of the desired particle size. The solvent/extract mixture may optionally be dried using conventional methods including, but not limited to, air drying, spray drying, freeze drying, etc. Moreover, the final product may be easily sterilized by filtration and infused into a variety of water-based products without changing their appearance. Such finished products will include CBD and THC-infused beverages (water, tea, coffee, beer, juice, etc.), creams, injections, oral and nasal sprays, tinctures, tablets, powders, edibles, thin film oral strips and many others. The final product may be stored at room temperature and may be further processed into pharmaceutical formulations and/or used for testing.

The all-in-one combination system is a convenient product that will tremendously simplify the ultrasonic production of high-quality, translucent nano-emulsions of bio-active ingredients such as cannabis extracts. This product is for manufacturers without the desire or capability to develop their own formulations and processing procedures. This product is designed to work in conjunction with a laboratory, bench or industrial ultrasonic processor, and comes with detailed, easy-to-follow instructions. It has practically no taste of its own and yields highly translucent and fully water-compatible nano-emulsions with droplet sizes less than 50 nanometers, ensuring a high bioavailability, accelerated onset of action, and permanent product stability.

The final SENE product may be formulated with any convenient pharmaceutically acceptable diluents, carriers or excipients to produce a pharmaceutical composition. The choice of diluents, carriers or excipients will depend on the desired dosage form, which may in turn be dependent on the intended route of administration to a patient. Oral dosage forms include, but are not limited to, tablets, capsules, suspensions, granules, oral depot gels, and solutions. The pharmaceutical preparations of the present invention are manufactured in a manner which is itself well known in the art. For example, the pharmaceutical preparations may be made by means of conventional mixing, granulating, dissolving, and lyophilizing processes. The processes to be used will depend ultimately on the physical properties of the active ingredient used.

Suitable excipients are, in particular, fillers such as sugars for example, lactose or sucrose, mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Oral dosage forms may be provided with suitable coatings which, if desired, may be resistant to gastric juices. For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the table coatings, for example, for identification or in order to characterize different combination of compound doses.

Other pharmaceutical preparations which can be used orally include capsules made of gelatin, as well as soft, sealed capsules made of gelatin and a plasticizer such as glycerol or sorbitol. The capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids; such as fatty oils, liquid paraffin, or liquid polyethylene glycols. In addition stabilizers may be added.

The pharmaceutical preparations of the present invention are manufactured in a manner which is itself well known in the art. For example the pharmaceutical preparations may be made by means of conventional mixing, granulating, dissolving, and lyophilizing processes. Such dosage forms may be prepared in accordance with standard principles of pharmaceutical formulation, known to those skilled in the art. The extract may be formulated for oral use (e.g. capsules) in dosage forms that provide 5 mg, 10 mg, 20 mg, or 100 mg of total cannabinoids per dose.

In another embodiment, the invention comprises an all-in-one surfactant product that can be sold distributed without the active component. The user can then later simply add the surfactant to the active ingredient along with water in the amounts and ratios already described above to provide a solubilized drug product.

The following examples are offered to illustrate but not limit the invention. Thus, it is presented with the understanding that various formulation modifications as well as method of delivery modifications may be made and still are within the spirit of the invention.

### Example 1

A pharmaceutical composition was prepared as described below. In one aspect, the invention comprises a pharmaceutical oral, tasteless, translucent nano-emulsion, consisting of a SENE comprising a mixture of the following:

| | | |
|---|---|---|
| Cannabis Extract | 1-10 | gm |
| Span 80 | 1-40 | gm |
| Ethoxy diglycol | 0.1-5 | gm |
| Polysorbate 80 | 1-40 | gm |
| Caprylic acid triglyceride | 5-85 | gm |
| Water | qs 100 | gm |

Procedure:

1. Combine all ingredients, mix for 15 minutes

2. Ultrasonicate for specific time with specific amplitude, filter

3. Test for particle size

4. Store in amber glass, at room temperature

### Example 2

A pharmaceutical composition was prepared as described below. In a second aspect, the invention comprises the all-in-one combination surfactant system, comprising a mixture of the following:

| | | |
|---|---|---|
| Span 80 | 1-40 | gm |
| Ethoxy diglycol | 0.1-5 | gm |
| Polysorbate 80 | 1-40 | gm |
| Caprylic Acid Triglyceride | 5-85 | gm |

Procedure:

1. Add all ingredients to beaker

2. Mix until homogeneous

3. Store in opaque plastic containers at room temperature

### Example 3

A pharmaceutical composition was prepared as described below. In one aspect, the invention comprises a pharmaceutical oral, tasteless, translucent nano-emulsion, consisting of a SENE comprising a mixture of the following:

| | | |
|---|---|---|
| Cannabis Extract | 1-10 | gm |
| Lecithin | 1-20 | gm |
| Kolliphor HS15 | 0.1-10 | gm |
| Polysorbate 80 | 1-20 | gm |
| Caprylic acid triglyceride | 5-85 | gm |
| Glycerine | 1-25 | gm |
| Water | qs 100 | gm |

Procedure:

5. Combine all ingredients, mix for 15 minutes

6. Ultrasonicate for specific time with specific amplitude, filter

7. Test for particle size

8. Store in amber glass, at room temperature, protected from light

### Example 4

A pharmaceutical all-in-one composition product was prepared as described below. In a second aspect, the invention comprises the all-in-one combination surfactant system, comprising a mixture of the following:

| | | |
|---|---|---|
| Lecithin | 1-25 | gm |
| Kolliphor HS15 | 0.1-20 | gm |
| Polysorbate 80 | 1-20 | gm |
| Glycerine | 1-40 | gm |
| Caprylic Acid Triglyceride | 5-85 | gm |

Procedure:

4. Add all ingredients to beaker

5. Mix until homogeneous

6. Store in opaque plastic containers at room temperature.

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplishes at least all of the intended objectives.

What is claimed is:

1. A hydrophilic, edible, emulsified drug delivery system (SENE) comprising: a solvent/stabilizer; and a combination of surfactants, said combination of surfactants having an overall HLB of between about 8-15.

2. The SENE of claim 1 whereby the surfactants are Polysorbate 80 and Span 80.

3. The SENE of claim 1 whereby the surfactants are lecithin and Polysorbate 80.

4. The SENE of claim 1 further comprising a vegetable or edible oil.

5. The SENE of claim 4 whereby the vegetable or edible oil is triglyceride oil.

6. The SENE of claim 5 whereby the triglyceride oil has an iodine value of between about 70 and 110.

7. The SENE of claim 5 whereby the triglyceride oil is selected from the group consisting of MCTs, LCTs, sunflower oil, maize germ oil, olive oil, avocado, and peanut oil.

8. The SENE of claim 5 whereby the triglyceride oil is caprylic acid triglceride oil.

9. The SENE of claim 1 whereby the solvent/stabilizer is selected from the group consisting of ethoxy glycol (EG), polyoxamer 188, and polyoxamer 407.

10. The SENE of claim 1 whereby the solvent/stabilizer is a hydroxystearate stabilizer.

11. The SENE of claim 10 whereby the solvent/stabilizer is Kolliphor HS15.

12. The SENE of claim 1 further including water.

13. The SENE of claim 1 further including a poorly water soluble medicament.

14. The SENE of claim 13 further including a cannabinoid.

15. The SENE of claim 1 that is translucent and has practically no taste.

16. The SENE of claim 1 having droplet sizes of less than 50 nanometers.

17. The SENE of claim 11 comprising up to 100 mg/ml of medicament.

18. A method of making a hydrophilic, edible, emulsified drug delivery system (SENE) comprising: combining a solvent/stabilizer with a combination of surfactants, said surfactants having an overall HLB of between about 8-15.

19. A method of making an all-in-one combination hydrophilic, edible, emulsified drug delivery system (SENE) to solubilize a poorly soluble medicament comprising combining a solvent/stabilizer with a combination of surfactants, said surfactants having an overall HLB of between about 8-15 to form the SENE; and combining the SENE with a poorly soluble drug and water to form a solubilized drug.

20. The method of claim 19 further including the step of infusing the SENE into a finished product selected from the group consisting of a beverage, cream, oral spray, nasal cream, tincture, injection, tablet, powder, edible, and thin film oral strips.
